(19) United States
(12) Patent Application Publication
(10) Pub. No.: US 2013/0180589 A1
(43) Pub. Date: Jul. 18, 2013

(54) ORGANIC PHOTOVOLTAIC CELL MATERIALS AND COMPONENTS

(71) Applicants: Feng-Wen YEN, Hsinchu County (TW); Cheng-Hao CHANG, Hsinchu County (TW); Chin-Min TENG, Hsinchu County (TW)

(72) Inventors: Feng-Wen YEN, Hsinchu County (TW); Cheng-Hao CHANG, Hsinchu County (TW); Chin-Min TENG, Hsinchu County (TW)

(73) Assignee: LUMINESCENCE TECHNOLOGY CORPORATION, Hsinchu County (TW)

(21) Appl. No.: 13/438,516

(22) Filed: Apr. 3, 2012

(30) Foreign Application Priority Data
Jan. 13, 2012 (TW) 101101446

(51) Int. Cl. H01L 51/46; C07D 487/04; C07D 495/04

(52) U.S. Cl. 136/263; 549/50; 548/418; 548/453

(57) ABSTRACT

The present invention relates to organic photovoltaic cell materials and components and particularly, to the organic photovoltaic cell materials and the components with high optical conversion efficiency, simple preparation process and low cost. The chemical formula of the materials is represented by chemical formula (I):

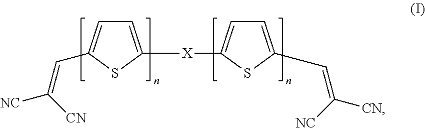

where n is a natural number and X is the following chemical formula (II):

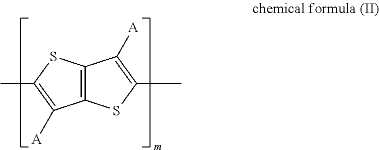

where m is 1˜3 and A is hydrogen, fluorine, chlorine, C₁˜C₁₈-alkyl, thienyl, phenyl or pyridyl in which thienyl, phenyl or pyridyl may be substituted with C₁˜C₁₈-alkyl in any position.

### BACKGROUND

1. Technical Field of the Invention

The present invention relates to organic photovoltaic cell materials and components and particularly, to the organic photovoltaic cell materials and components with high optical conversion efficiency, simple preparation process and low cost.

2. Description of Related Art

The application of solar energy, light energy, etc. to the electric power generation has become one of main developing technology in the fields of the green energy and the environmental protection now. Due to the lack of the earth's energy resources, people focus on the inexhaustible energy resources such as solar energy, wind power generation, etc. available from the nature gradually. Further, the development of the displayer focuses on the conversion of a thin structure to a portable and flexible thin structure. Thus, the supply of electric power resources to a thinning displayer also becomes one of main research subjects.

In order to achieve the foregoing needs, the photovoltaic cells were brought into markets. Currently, the types of the photovoltaic cells are classified into inorganic and organic photovoltaic cells, wherein the potential of organic photovoltaic cells is more noticeable. For the organic photovoltaic cells, components with a broader solar spectrum are produced by the adjustment of the band gap of organic materials through chemical synthesis. Besides, the components have a high light-absorption coefficient and thus, the thickness of the organic layer only requires several hundred nanometers. Furthermore, the tandem cell can be used to design materials that can absorb the specific wave length of the spectra, whereby the conversion efficiency is raised.

The organic photovoltaic cells are produced mainly from an organic material having a semiconductor property. The advantages of this process are lower production costs, lighter materials, the structural designability of compounds, the producibility of photovoltaic cells having a large surface, the production in large scale, excellent processability, high light-absorption coefficient and the properties such as flexibility, semi-transparency, etc. However, there are problems such as low power conversion efficiency, low carrier mobility, high electrical resistance, poor durability, etc. to be overcome now.

According to the different properties of the organic materials, the types of the organic photovoltaic cells can be further classified into (1) dye-sensitized solar cells, (2) small molecular solar cells and (3) polymer solar cells. Among these, the commonly used materials of the polymer solar cells include poly(3-hexylthiophene) (P3HT) and phenyl-C61-butyric acid methyl ester (PCBM) (n type material). Further, the ordinary process is to dissolve these two organic semiconductor materials in a solvent followed by mixing and application to a component. After well mixing, the surface of the pn interface can be effectively increased, and the opportunity of the separation of excitons increases, thereby resulting in the enhancement of the efficiency of cells.

US patent laid-open No. 2009/0217980A1 discloses an organic photovoltaic component and an organic material used thereof. However, the photon-to-electron conversion efficiency of the photovoltaic cell component is not high when the said material is used therein. Further, the production yield of the material is not high and thus, the cost cannot be effectively reduced. Moreover, the complex process of the said component is also one of main reasons that cannot effectively reduce the cost.

### SUMMARY

In view of the above drawbacks of the materials of the photovoltaic cell components, the inventors actively engage themselves in research and development in order to improve the above, drawbacks of the conventional structures. After continued efforts and experimentation, the present invention was developed finally.

The main object of the present invention is to provide an organic photovoltaic cell material and component which have the advantages of high photon conversion efficiency, simple preparation process and low cost.

In order to achieve the above object of the invention, the present invention adopots the following technical means. Among these, the organic photovoltaic cell materials are represented by chemical formula (I):

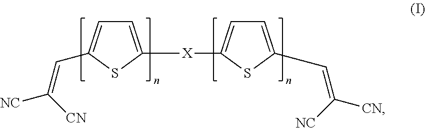

where n is a natural number and X is the following chemical formula (II):

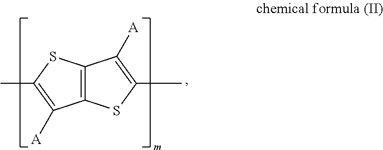

where in is 1˜3 and A is hydrogen, fluorine, chlorine, C₁˜C₁₈-alkyl, thienyl, phenyl or pyridyl, in which thienyl, phenyl or pyridyl may be substituted with C₁˜C₁₈-alkyl in any position.

Preferably, X is any one of the following chemical formulae (III), (IV) and (V):

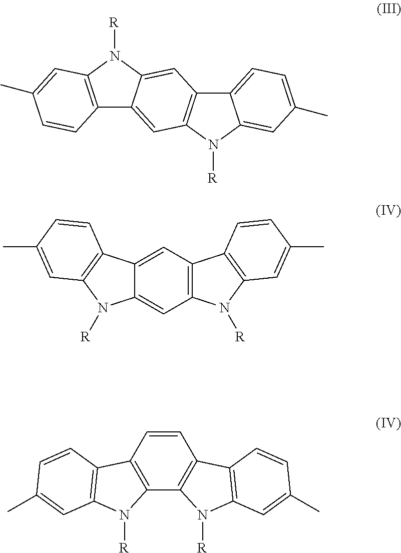

where R is C₁˜C₁₈-alkyl, phenyl which is substituted with C₁˜C₁₈-alkyl in any position, or phenyl.

Preferably, X is the following chemical formula (VI):

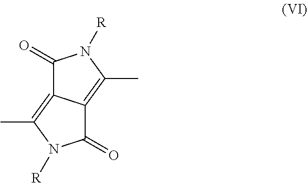

where R is C₁˜C₁₈-alkyl, phenyl which is substituted with C₁˜C₁₈-alkyl in any position, or phenyl.

Preferably, n is 1˜4.

Besides, the organic photovoltaic cell component of the invention comprises an electron-donor layer, at least one electron-acceptor layer and an electron transport layer (or an hole/exciton barrier layer) which are attached to a substrate by the thermal evaporation or the spin coating in turn and are arranged between an anode and a cathode.

The electron-donor layer contains the forging organic photovoltaic cell materials of the invention.

The organic photovoltaic cell components with the compounds of the invention shown by the chemical formula (I) as the electron-donor layer have advantages such as the high photon-to-electron conversion efficiency, the simple structure and the reduced cost by virtue of the high yields. The photon-to-electron conversion efficiency thereof can be close to 6%.

### BRIEF DESCRIPTION OF THE DRAWING

FIG. 1 shows a TGA diagram of Compound I.

FIG. 2 shows the J-V test result of Component of Example 1 having Compound I as an electron donor layer and 2,9-dimethyl-4,7-diphenyl-1,10-phenanthroline as an electron transport layer.

FIG. 3 shows the J-V test result of Component of Example 1 having Compound I as an electron donor layer and 4,7-diphenyl-2,9-bis(4-(1-phenyl-1H-benzo[d]imidazol-2-yl)phenyl)-1,10-phenanthroline as an electron transport layer.

FIG. 4 shows the J-V test result of Component of Example 4 having Compound XIV as an electron donor layer and 2,9-dimethyl-4,7-diphenyl-1,10-phenanthroline as an electron transport layer.

FIG. 5 shows the J-V test result of Component of Example 4 having Compound XIV as an electron donor layer and 4,7-diphenyl-2,9-bis(4-(1-phenyl-1H-benzo[d]imidazol-2-yl)phenyl)-1,10-phenanthroline as an electron transport layer.

### DETAILED DESCRIPTION

The organic photovoltaic cell component of the invention comprises an electron-donor layer, at least one electron-acceptor layer and an electron transport layer (or an electron/exciton barrier layer) attached to a substrate by the thermal evaporation or the spin coating in turn, which are arranged between an anode and a cathode.

When a component is irradiated by the light, a donor firstly accepts the light and then the electron-hole pair or the so-called exciton is form by the photo-irradiation. The exciton will decompose into independent conductive electron and hole when it diffuses to the interface of the donor and the acceptor. Further, due to the difference between LUMO and HUMO energy levels of both the donor and the acceptor, the electron will be transported to the acceptor material whereas the hole will be transported to the donor material. The current is then generated by the electrodes via an external circuit.

The embodiments of the organic materials of the present invention used in the electron-donor layer, which are represented by the above chemical formula (I), are illustrated as below. However, the relative compounds of the chemical formula (I) are not limited to the following embodiments.

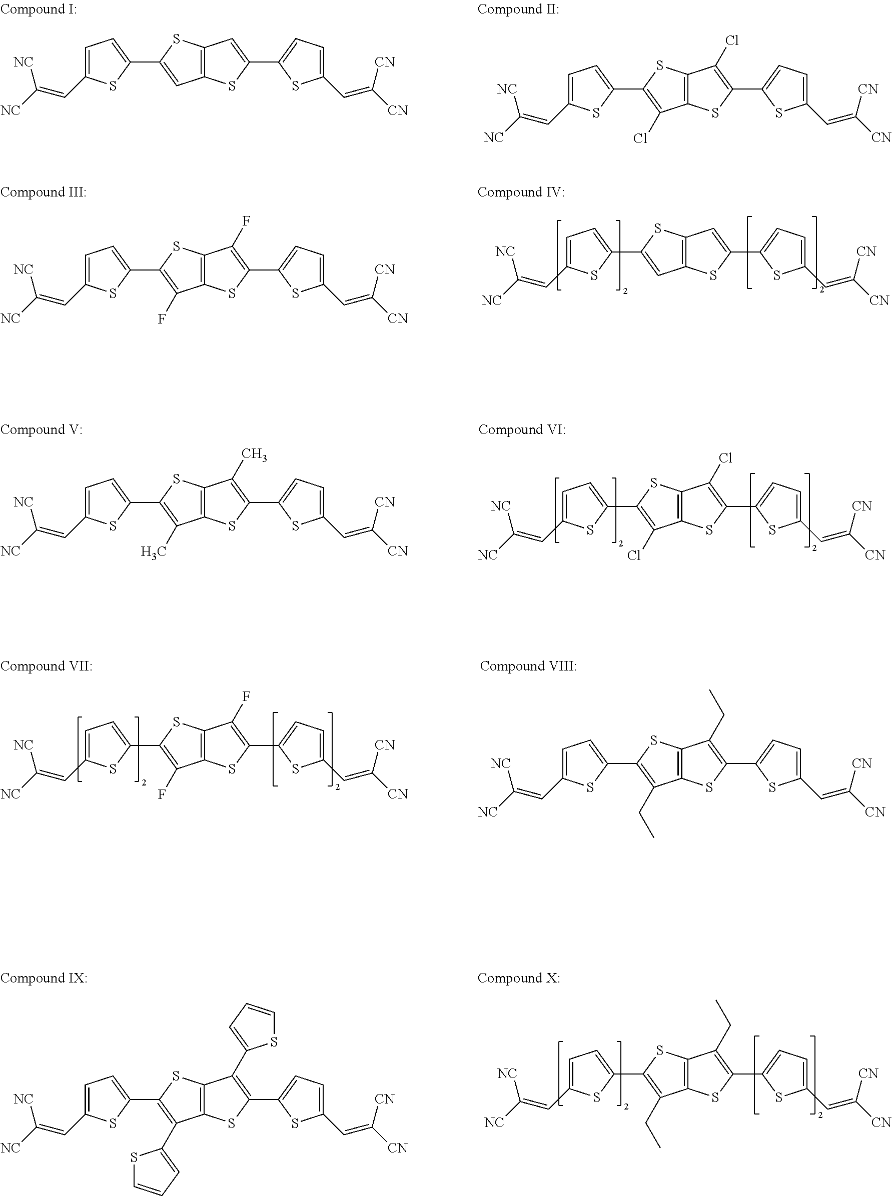

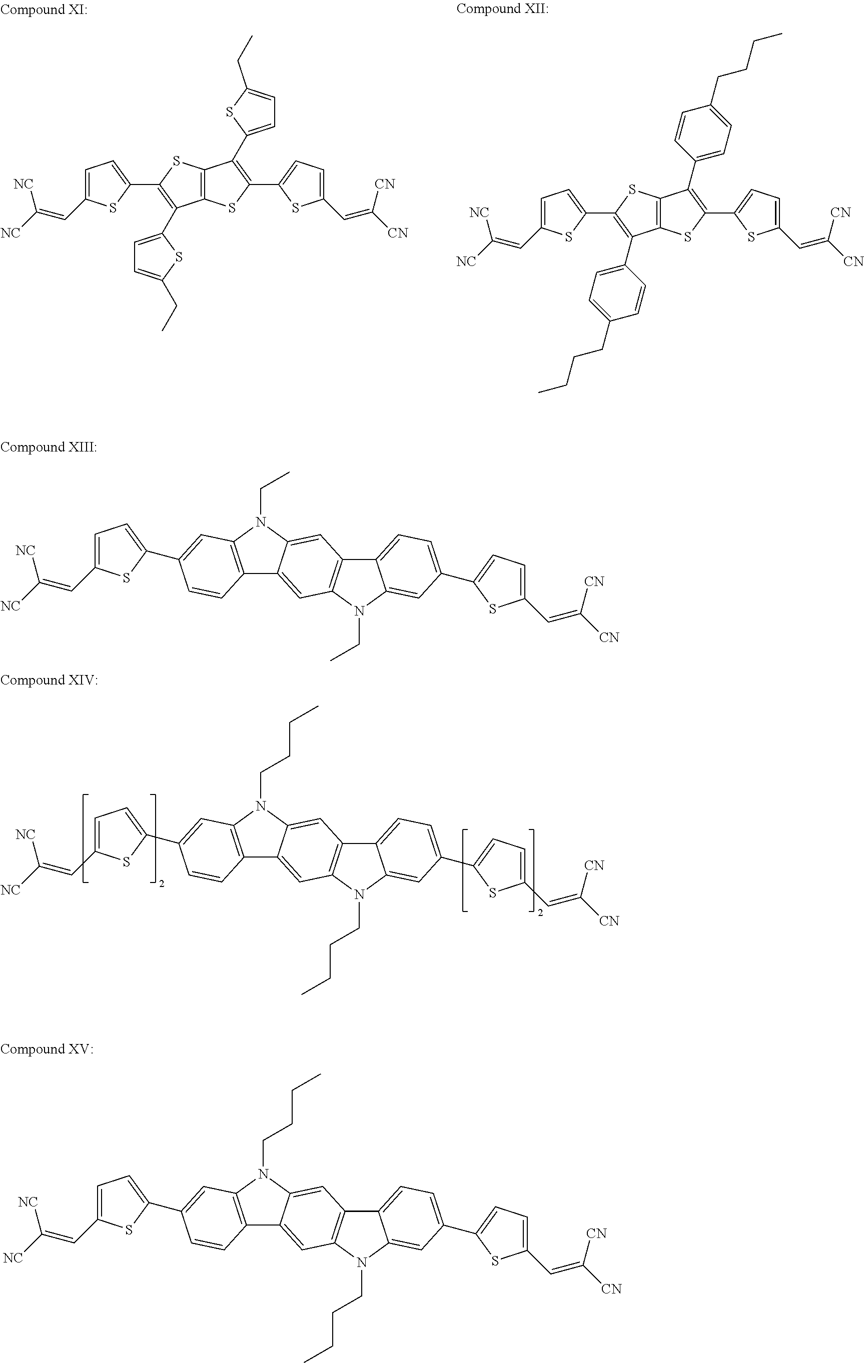

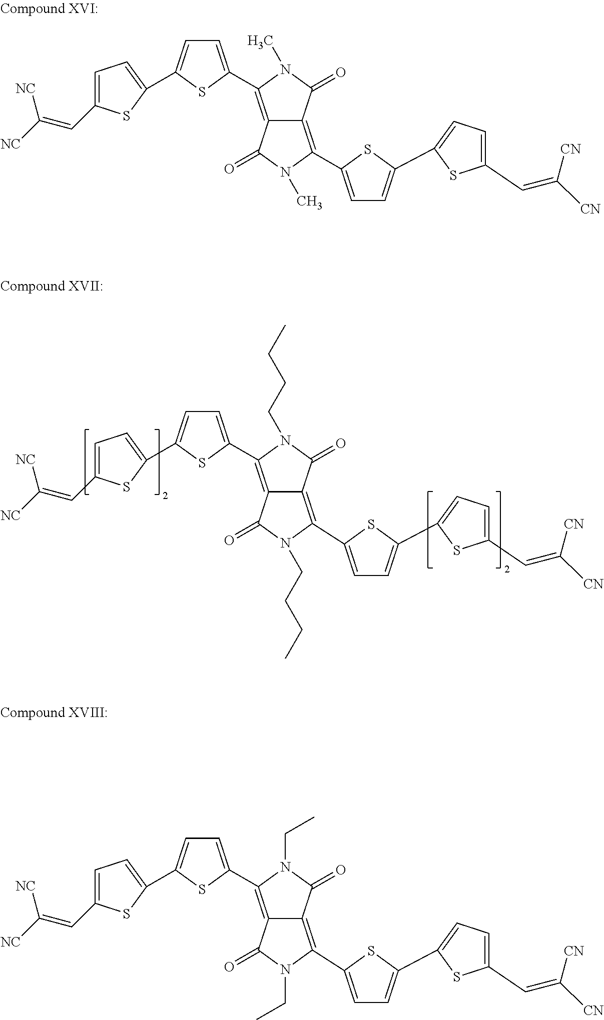

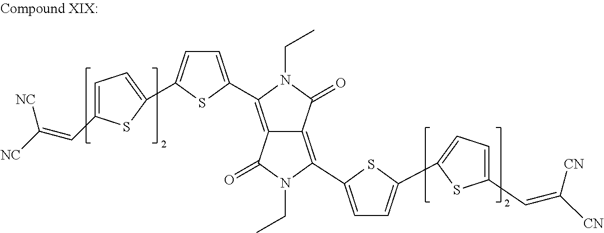

The preparations of the compounds of the present invention are illustrated by the examples as below.

### EXAMPLES

### The Synthesis Example 1

### The Preparation of the Compound I

The compound I can be prepared in accordance with the following reaction schemes (A) to (C):

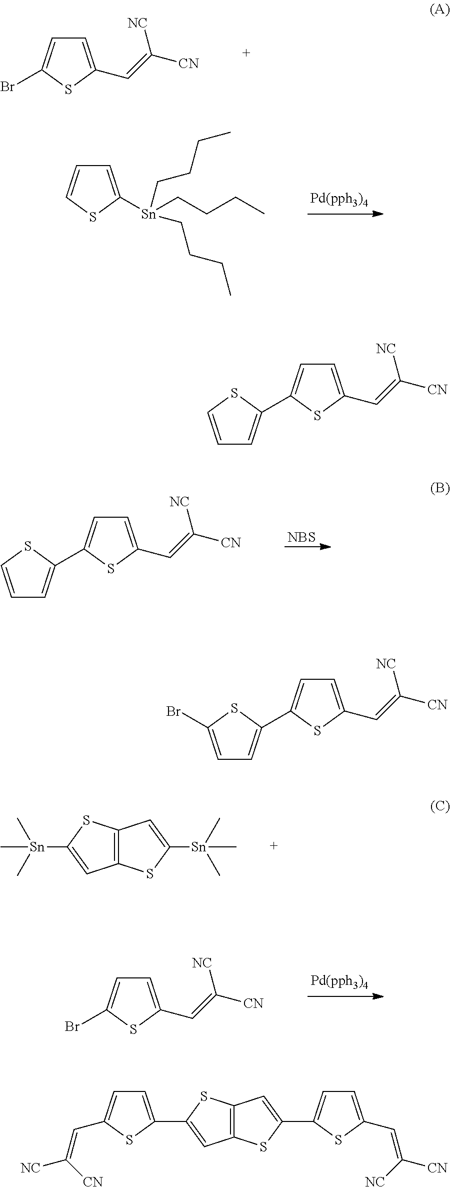

### The synthesis of 2-(2,2′-bithiophen-5-ylmethylene)malononitrile

2-((5-bromothiophen-2-yl)methylene)malononitrile (23.9 g, 0.1 mol), tributyl(thiophen-2-yl)stannane (44.7 g, 0.12 mol), a catalyst Pd(pph₃)₄ (1.15 g, 1 mmol) were added to toluene (240 mL) and mixed. The mixture was stirred at 110° C. for 18 hours under nitrogen gas and then cooled to room temperature. Subsequently, the reaction product was filtered and washed with methanol to give 18.1 g of 2-(2,2′-bithiophen-5-ylmethylene)malononitrile as an orange solid product (Yield: 75%). ¹H NMR (500 MHz, CDCl₃) δ (ppm) 8.64 (s, 1H), 7.89 (d, 1H), 7.78 (dd, 1H), 7.67 (dd, 1H), 7.62 (d, 1H), 7.18-7.22 (m, 1H).

### The synthesis of 2-((5′-bromo-2,2′-bithiophen-5-yl)methylene)malononitrile

2-(2,2′-bithiophen-5-ylmethylene)malononitrile (18.1 g, 74.69 mmol) and N-Bromo-succinimide (NBS) (13.95 g, 78.43 mmol) were added to dimethylformamide (DMF) (200 mL). The mixture was stirred at room temperature in dark for 24 hours under nitrogen gas. Subsequently, the reaction resultant was filtered and washed with methanol to give 18.1 g of 2-((5′-bromo-2,2′-bithiophen-5-yl)methylene)malononitrile as an orange solid product, (Yield: 75%). ¹H NMR (500 MHz, CDCl₃): δ (ppm) 8.64 (s, 1H), 7.88 (d, 1H), 7.61 (d, 1H), 7.51 (d, 1H), 7.34 (d, 1H), 7.35 (d, 1H).

### The Synthesis of the Compound I

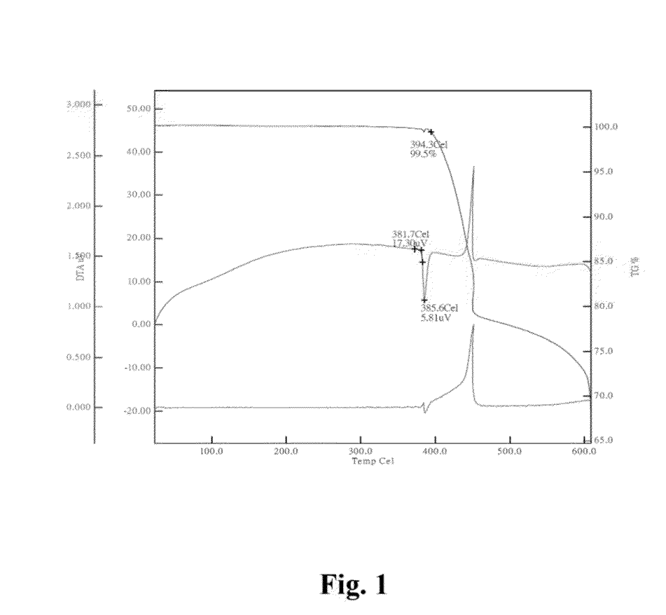

2,5-bis(trimethylstannyl)thieno[3,2-b]thiophene (4.65 g, 0.01 mol), 2-((5-bromothiophen-2-yl)methylene)malononitrile (5.73 g, 0.024 mol) and a catalyst Pd(pph₃)₄ (0.11 g, 0.1 mmol) were added to toluene (232 mL). The mixture was stirred at 110° C. for 18 hours under nitrogen gas. After cooling to room temperature, the reaction product was filtered and washed several times with acetone, hexane and methanol, respectively to give 4.2 g of the compound I as a greenish black solid (Yield: 92%; mp: 385° C.). ¹H NMR (500 MHz, d₆-DMSO) δ (ppm) 8.57 (s, 1H), 8.06 (s, 1H), 7.94 (d, 1H), 7.67 (d, 1H). FIG. 1 shows a TGA plot of compound I obtained by using a Diamond TG/DTA type thermogravimetris analyzer from Perkin Elmer.

### The Synthesis Example 2

### The Preparation of the Compound IV

The compound IV can be prepared in accordance with the following reaction scheme (D):

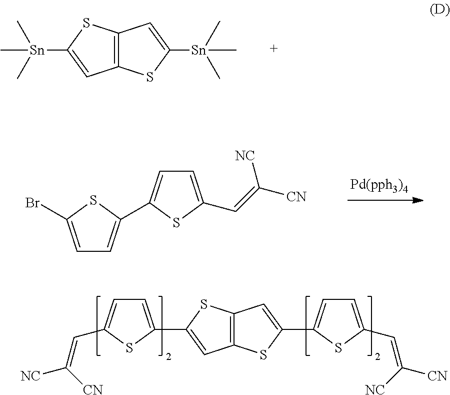

2,5-bis(trimethylstannyl)thieno[3,2-b]thiophene (4.65 g, 0.01 mol), 2-((5′-bromo-2,2′-bithiophen-5-yl)methylene)malononitrile (7.71 g, 0.024 mol) and a catalyst Pd(pph₃)₄ (0.11 g, 0.1 mmol) were added to dimethylformamide (DMF) (232 mL). The mixture was stirred at 110° C. for 18 hours under nitrogen gas. After cooling to room temperature, the reaction product was filtered and washed several times with acetone, hexane and methanol, respectively to give 5.9 g of the compound IV as a black solid (Yield: 96%; mp: 355° C.). EI-MS m/z(%)=620 (M⁺, 100%).

### The Synthesis Example 3

### The Preparation of the Compound XI

The compound XI can be prepared in accordance with the following reaction schemes (E) to (G):

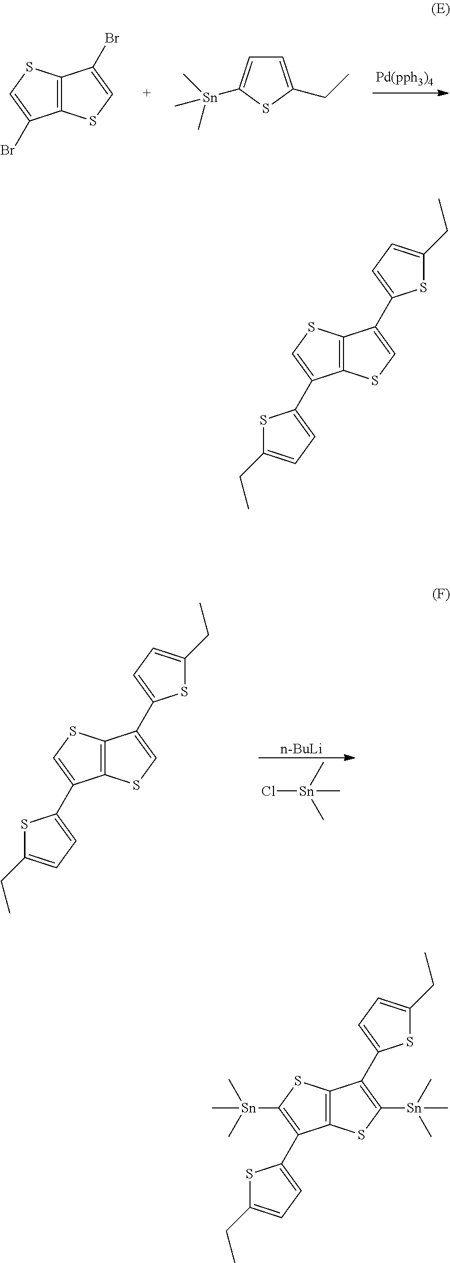

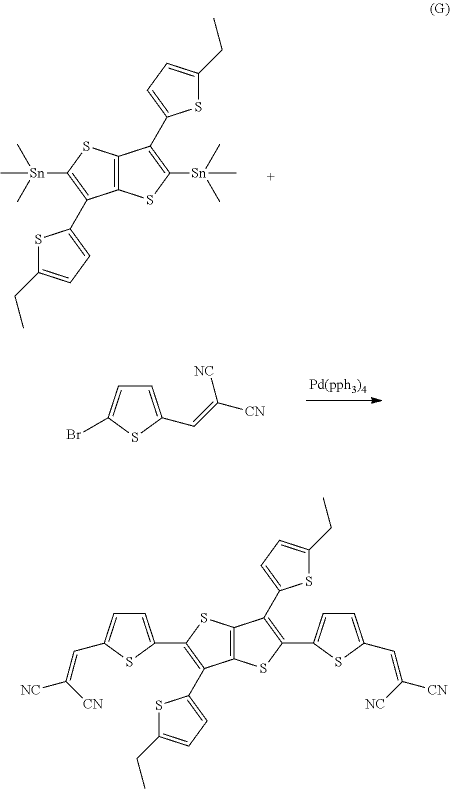

### The synthesis of 3,6-bis(5-ethylthiophen-2-yl)thieno[3,2-b]thiophene

3,6-dibromothieno[3,2-b]thiophene (14.9 g, 0.05 mol), (5-ethylthiophen-2-yl)trimethylstannane (41.2 g, 0.15 mol) and a catalyst Pd(pph₃)₄ (0.58 g, 0.5 mmol) were added to toluene (450 mL). The mixture was stirred at 110° C. for 24 hours under nitrogen gas. After cooling to room temperature, the reaction mixture was extracted with dichloromethane, washed with pure water and then dried with anhydrous sodium sulphate, followed by the purification by the column chromatography (dichloromethane: hexane=1:8) to give 12.6 g of a pale yellow solid (Yield: 70%). ¹H NMR (500 MHz, CDCl₃) δ (ppm) 7.31 (d, 2H), 7.28 (s, 2H), 6.83 (d, 2H), 2.87 (m, 4H), 1.25 (m, 6H); EI-MS: m/z(%)=688 (M⁺, 100%).

### The synthesis of 3,6-bis(5-ethylthiophen-2-yl)thieno[3,2-b]thiophene-2,5-diyl)bis(trimethylstannane)

3,6-bis(5-ethylthiophen-2-yl)thieno[3,2-b]thiophene (18.03 g, 0.05 mol) was added to tetrahydrofuran (THF) (540 mL). N-butyllithium (2.5M in hexane solution) (10.36 ml, 0.11 mol) was added dropwise to the foregoing solution at −78° under nitrogen gas, and stirred 1 hour at the same temperature and stirred for another 1 hour at room temperature. Then trimethyl tinchloride (23.91 g, 0.12 mol) was added at −78°. The mixture was stirred at room temperature for 24 hours. The reaction mixture was quenched with water, extracted twice with ethyl acetate, subsequently dried with anhydrous sodium sulphate, and finally purified by recrystallization with ethanol to give 24.36 g of a white solid (Yield: 71%). ¹H NMR (500 MHz, CDCl₃) δ (ppm) 7.28 (s, 2H), 6.83 (d, 2H), 2.87 (m, 4H), 1.25 (m, 6H), 0.42 (s, 12H); EI-MS: m/z(%)-676 (M⁺, 100%).

### The Synthesis of the Compound XI

The foregoing white solid (13.72 g, 0.02 mol), 2-((5-bromothiophen-2-yl)methylene)malononitrile (11.47 g, 0.048 mol) and a catalyst Pd(pph₃)₄ (0.23 g, 0.2 mmol) were added to dimethylformamide (DMF) (1372 mL). The mixture was stirred at 110° C. for 18 hours under nitrogen gas. After cooling to room temperature, the reaction mixture was filtered and washed several times with acetone, hexane and methanol, respectively to give 9.2 g of the compound XI as a black solid (Yield: 68%).

### The Synthesis Example 4

### The Preparation of the Compound XIV

The compound XIV can be prepared in accordance with the following reaction scheme (F):

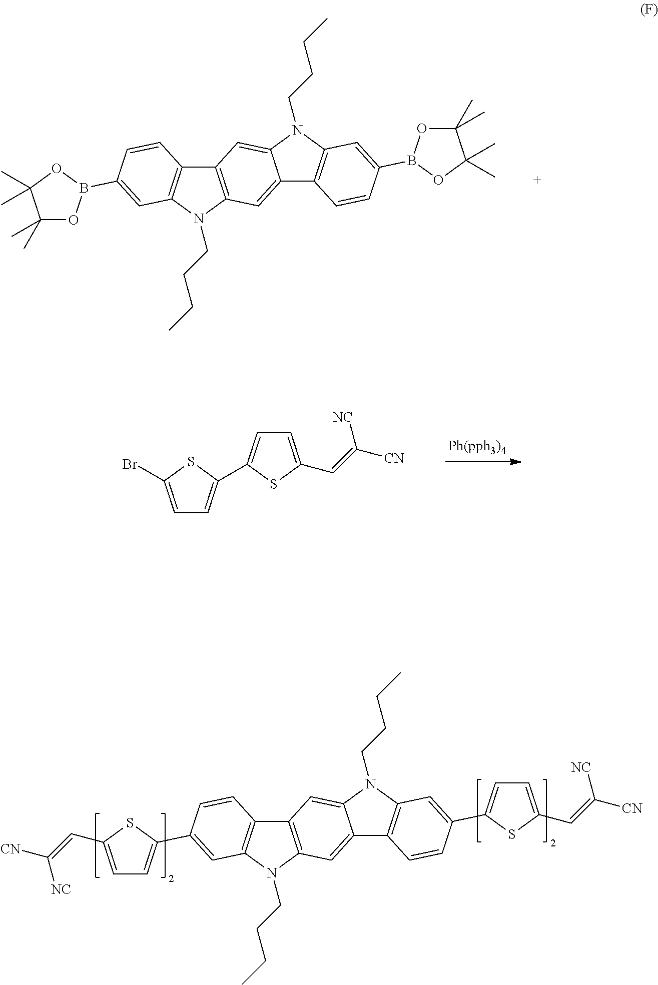

5,11-dibutyl-3,9-bis(4,4,5,5-tetramethyl-1,3,2-dioxaborolan-2-yl)-5,11-dihydroindolo[3,2-b]carbazole (6.20 g, 0.01 mol), 2-((5′-bromo-2,2′-bithiophen-5-yl)methylene)malononitrile (7.7 g, 0.024 mol), 2M Na₂CO₃(aq) (20 ml), and a catalyst Pd(pph₃)₄ (0.11 g, 0.1 mmol) were added to toluene (310 mL) and ethanol (155 ml) and mixed. The mixture was stirred at 90-100° C. for 24 hours under nitrogen gas. After cooling to room temperature, the reaction mixture was filtered and washed several times with acetone, hexane and methanol, respectively to give 4.58 g of the dark red solid (Yield: 54%).

### The Synthesis Example 5

### The Preparation of the Compound XVI

The compound XVI can be prepared in accordance with the following reaction schemes (H) and (I):

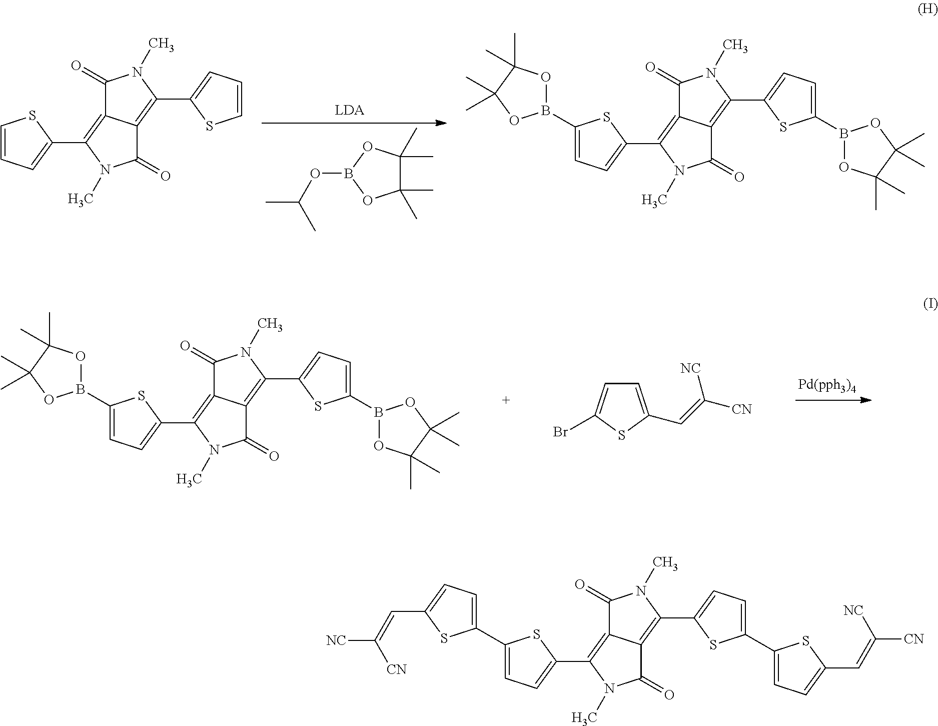

### The synthesis of 2,5-dimethyl-3,6-bis(5-(4,4,5,5-tetramethyl-1,3,2-dioxaborolan-2-yl)thiophen-2-yl)pyrrolo[3,4-c]pyrrole-1,4(2H,5H)-dione

2,5-dimethyl-3,6-di(thiophen-2-yl)pyrrolo[3,4-c]pyrrole-1,4(2H,5H)-dione (16.42 g, 0.05 mol) was added to tetrahydrofuran (THF) (145 mL). Lithium diisopropylamide (10 wt. % in hexane) (107.1 g, 0.1 mol) was added dropwise at 0° C. under nitrogen gas, and stirred for 1 hour. Then, 2-isopropoxy-4,4,5,5-tetramethyl-1,3,2-dioxaborolane (22.32 g, 0.12 mol) was added dropwise at −78° C. The mixture was stirred at room temperature for 24 hours. The reaction mixture was quenched with water, extracted twice with ethyl acetate, subsequently dried with anhydrous sodium sulphate, and finally purified by recrystallization with ethanol to give 12.4 g of the solid product (Yield: 43%). ¹H NMR (500 MHz, CDCl₃) δ (ppm) 8.62 (d, 2H), 7.20 (d, 2H), 3.92 (m, 6H), 1.31 (s, 24H); EI-MS m/z(%)=644 (M⁺, 100%).

### The Synthesis of the Compound XVI

2,5-dimethyl-3,6-bis(5-(4,4,5,5-tetramethyl-1,3,2-dioxaborolan-2-yl)thiophen-2-yl)pyrrolo[3,4-c]pyrrole-1,4(2H,5H)-dione (2.9 g, 0.005 mol), 2-((5-bromothiophen-2-yl)methylene)malononitrile (2.86 g, 0.012 mol), 2M Na₂CO₃(aq) (10 ml) and a catalyst Pd(pph₃)₄ (0.058 g, 0.05 mmol) were added to dimethylformamide (DMF) (145 mL). The mixture was stirred at 90-100° C. for 24 hours under nitrogen gas. After cooling to room temperature, the reaction mixture was filtered and washed several times with dichloromethane/methanol to give 2.16 g of the compound XVI as a dark blue solid (Yield: 88%). EI-MS m/z(%)=644 (M⁺, 100%).

It can be known from each of the foregoing synthesis examples that the maximum synthesis yield of the materials of the present invention can be higher than 90%. Thus, the production costs of the materials can be effectively reduced and thus, the overall costs of the components are reduced.

### The Examples of the Component

### The Preparation Method of the Component

An indium tin oxide (ITO) glass substrate having a surface resistance of <15Ω in dimension of 50 mm×50 mm×0.7 mm (thickness) (commercially available from BUWON ACT Co., Ltd.) was prepared. Before use, the ITO substrate needs to be immersed into isopropyl alcohol and acetone solutions in turn, washed by ultrasound for 5 mins, washed by the UV ozone and then immediately deposited to an evaporator, followed by evacuation to 3×10⁻⁷ Torr.

The evaporation of an electron-donor layer with 200 Å of the thickness was carried out by the materials of the present invention on the ITO substrate. Subsequently, the evaporation of fullerene (C60) (commercially available from SES Research) as an electron-acceptor layer with 500 Å was carried out at the evaporation rate of 1 Å/s on the electron-donor layer. After that, the evaporation of an electron transport layer (or a hole/exciton barrier layer) with the thickness of 60 Å was carried out at the evaporation rate of 0.5 Å/s on the electron-donor layer, wherein 2,9-dimethyl-4,7-diphenyl-1,10-phenanthroline (commercially available from Lumtec.) or 4,7-diphenyl-2,9-bis(4-(1-phenyl-1H-benzo[d]imidazol-2-yl)phenyl)-1,10-phenanthroline (commercially available from Lumtec.) was used as the material of the electron transport layer. Finally, the evaporation of the metal (aluminum, commercially available from Well-Being Enterprise Co.) with the thickness of 1600 Å as cathode was carried out at the evaporation rate of 10 Å/s on the electron transport layer (or the hole/exciton barrier layer) and the surface area of the component was controlled to 0.09 cm² with a mask. After the evaporation, the component was packaged directly in the body of the evaporator under the nitrogen gas atmosphere.

J-V Test (Current Density Versus Voltage Characteristics):

Test method: the experimental data of the photon-to-electron conversion efficiency was determined by the irradiation of AM 1.5G simulating the sun light of 100 mW/cm² (lsun) at a constant temperature and a dark environment.

### Example 1

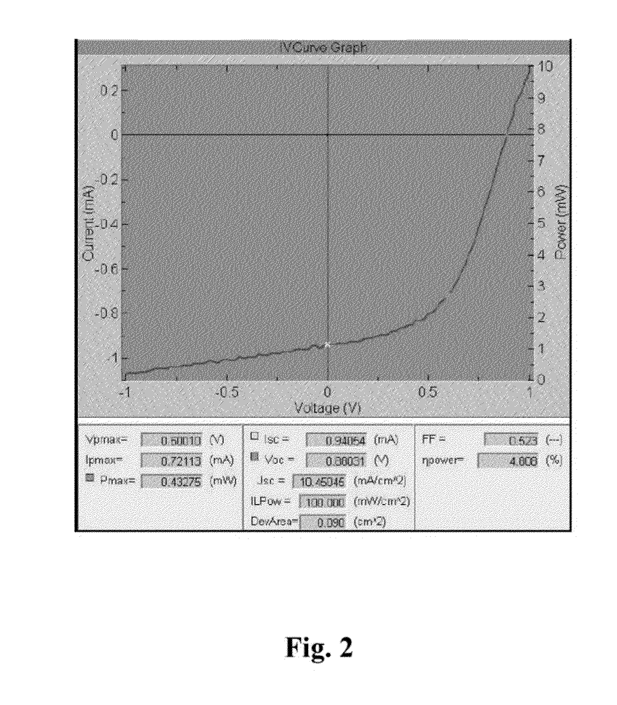

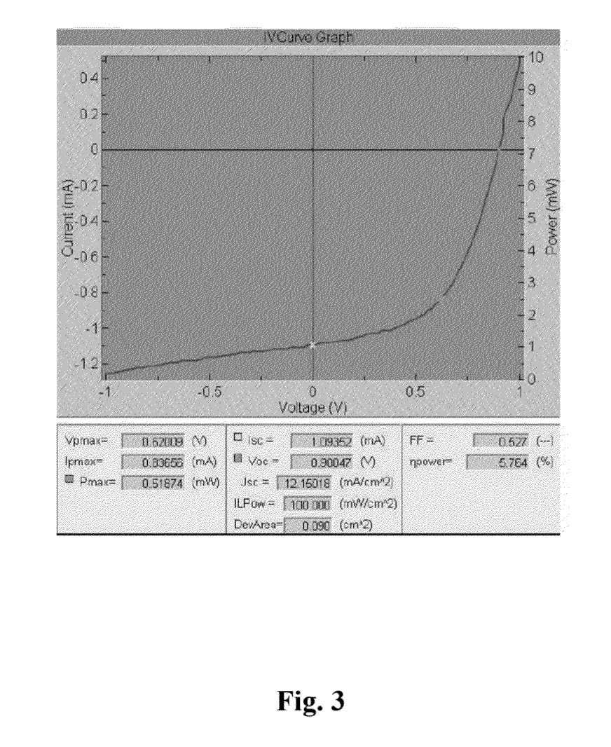

### Compound I was used as the material of the electron-donor layer of the component (organic material layer). When 2,9-dimethyl-4,7-diphenyl-1,10-phenanthroline was used as an electron transport layer between an electron acceptor layer and cathode, the photon-to-electron conversion efficiency was 4.808%. When 4,7-diphenyl-2,9-bis(4-(1-phenyl-1H-benzo[d]imidazol-2-yl)phenyl)-1,10-phenanthroline was used as an electron transport layer, the photon-to-electron conversion efficiency was 5.764%. The foregoing test results are shown in FIGS. 2 and 3, wherein the electron transport layer uses 2,9-dimethyl-4,7-diphenyl-1,10-phenanthroline in FIG. 2 and the electron transport layer uses 4,7-diphenyl-2,9-bis(4-(1-phenyl-1H-benzo[d]imidazol-2-yl)phenyl)-1,10-phenanthroline in FIG. 3.

### Example 2

Compound IV was used as the material of the electron-donor layer of the component (organic material layer). When 2,9-dimethyl-4,7-diphenyl-1,10-phenanthroline was used as an electron transport layer between an electron acceptor layer and cathode, the photon-to-electron conversion efficiency was 2.427%. When 4,7-diphenyl-2,9-bis(4-(1-phenyl-1H-benzo[d]imidazol-2-yl)phenyl)-1,10-phenanthroline was used as an electron transport layer, the photon-to-electron conversion efficiency was 3.018%.

### Example 3

Compound XI was used as the material of the electron-donor layer of the component (organic material layer). When 2,9-dimethyl-4,7-diphenyl-1,10-phenanthroline was used as an electron transport layer between an electron acceptor layer and cathode, the photon-to-electron conversion efficiency was 0.927%. When 4,7-diphenyl-2,9-bis(4-(1-phenyl-1H-benzo[d]imidazol-2-yl)phenyl)-1,10-phenanthroline was used as an electron transport layer, the photon-to-electron conversion efficiency was 1.398%.

### Example 4

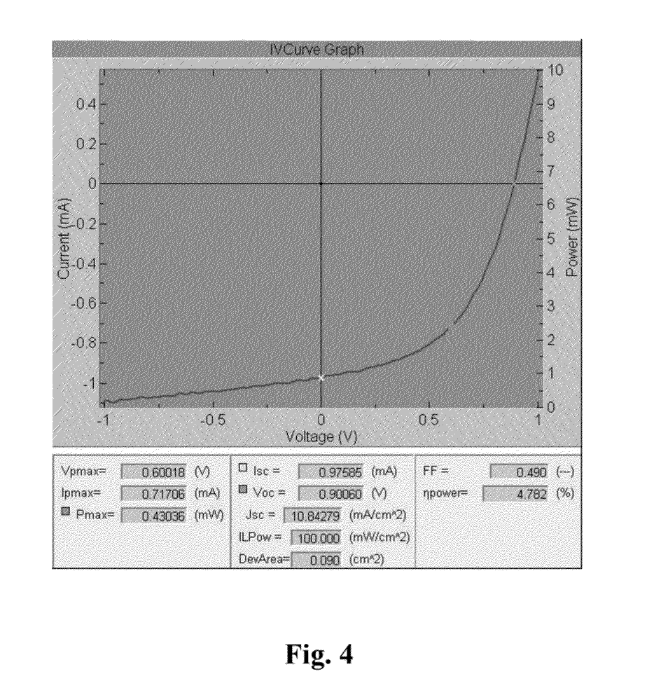

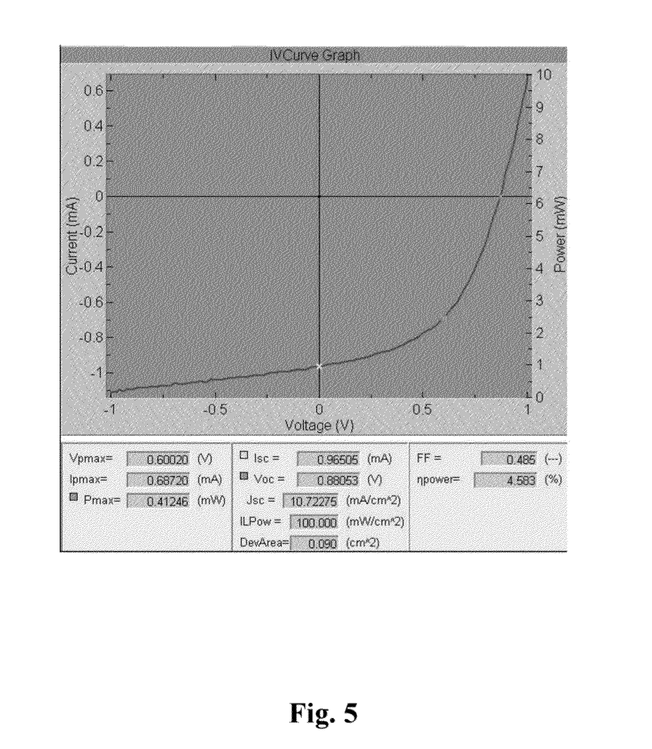

Compound XIV was used as the material of the electron-donor layer of the component (organic material layer). When 2,9-dimethyl-4,7-diphenyl-1,10-phenanthroline was used as an electron transport layer between an electron acceptor layer and cathode, the photon-to-electron conversion efficiency was 4.782%. When 4,7-diphenyl-2,9-bis(4-(1-phenyl-1H-benzo[d]imidazol-2-yl)phenyl)-1,10-phenanthroline was used as an electron transport layer, the photon-to-electron conversion efficiency was 4.583%. The foregoing test results are shown in FIGS. 4 and 5, wherein the electron transport layer uses 2,9-dimethyl-4,7-diphenyl-1,10-phenanthroline in FIG. 4 and the electron transport layer uses 4,7-diphenyl-2,9-bis(4-(1-phenyl-1H-benzo[d]imidazol-2-yl)phenyl)-1,10-phenanthroline in FIG. 5.

### Example 5

Compound XVI was used as the material of the electron-donor layer of the component (organic material layer). When 2,9-dimethyl-4,7-diphenyl-1,10-phenanthroline was used as an electron transport layer between an electron acceptor layer and cathode, the photon-to-electron conversion efficiency was 1.330%. When 4,7-diphenyl-2,9-bis(4-(1-phenyl-1H-benzo[d]imidazol-2-yl)phenyl)-1,10-phenanthroline was used as an electron transport layer, the photon-to-electron conversion efficiency was 1.386%.

### Comparative Example

The material of the following chemical formula (DCV5T) disclosed in US patent laid-open No. 2009/0217980 A1 was used as the material of the electron-donor layer (organic material layer). When 2,9-dimethyl-4,7-diphenyl-1,10-phenanthroline was used as an electron transport layer between an electron acceptor layer and cathode, the photon-to-electron conversion efficiency was 0.826%.

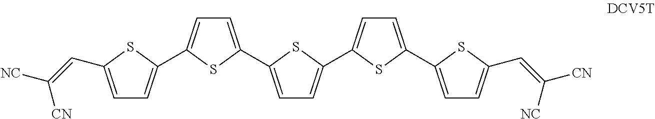 [SMILES: N#CC(C#N)=Cc1ccc(-c2ccc(-c3ccc(-c4ccc(-c5ccc(C=C(C#N)C#N)s5)s4)s3)s2)s1]

It can be understood from the foregoing comparative example and examples of the components that not only the yields of the materials of the present invention are very high but also for the components of the present invention, only evaporation of a three-layer structure between the anode and the cathode is required to achieve effective photon-to-electron conversion efficiency. Such structure is different from the five-layer structure disclosed in US patent laid-open No. 2009/0217980 A 1 (For example, Example 3 of the said prior art). The production of the structure for the components of the present invention is simpler and thus, the production cost of the components can be effectively reduced and the commercial competitiveness can be enhanced.

1. An organic photovoltaic cell material, which is represented by chemical formula (I): 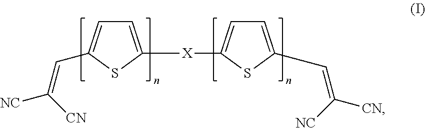
where n is a natural number and
X is the following chemical formula (II): 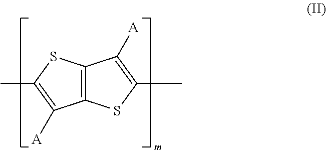
where m is 1˜3 and A is hydrogen, fluorine, chlorine, C₁˜C₁₈-alkyl, thienyl, phenyl or pyridyl, in which thienyl, phenyl or pyridyl may be substituted with C₁˜C₁₈-alkyl in any position; or
any one of the following chemical formulae (III), (IV), (V) and (VI): 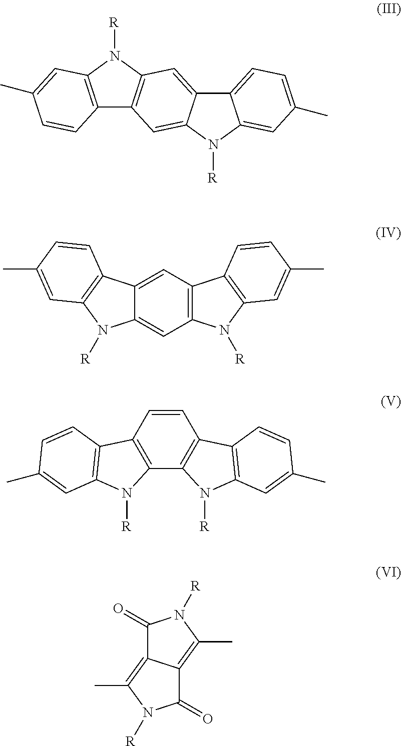
where R is C₁˜C₁₈-alkyl, phenyl which is substituted with C₁˜C₁₈-alkyl in any position or phenyl.

2. The organic photovoltaic cell material according to claim 1, wherein n is 1˜4.

3. The organic photovoltaic cell material according to claim 1, wherein the material is

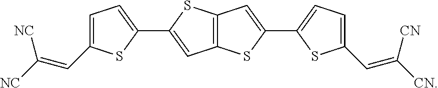 [SMILES: N#CC(C#N)=Cc1ccc(-c2cc3sc(-c4ccc(C=C(C#N)C#N)s4)cc3s2)s1]

4. The organic photovoltaic cell material according to claim 1, wherein the material is

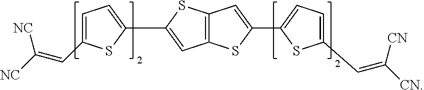

5. The organic photovoltaic cell material according to claim 1, wherein the material is

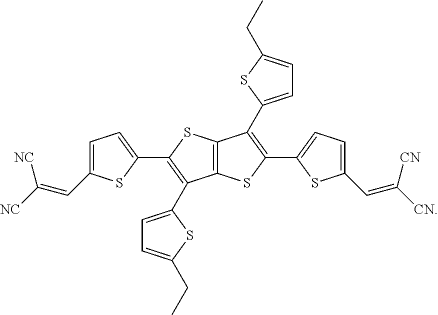 [SMILES: CCc1ccc(-c2c(-c3ccc(C=C(C#N)C#N)s3)sc3c(-c4ccc(CC)s4)c(-c4ccc(C=C(C#N)C#N)s4)sc23)s1]

6. The organic photovoltaic cell material according to claim 1, wherein the material is

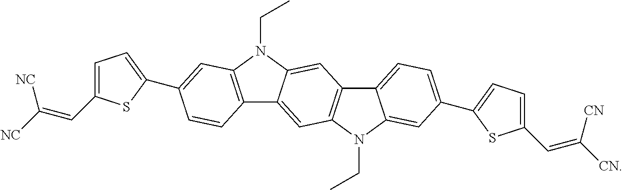 [SMILES: CCn1c2cc(-c3ccc(C=C(C#N)C#N)s3)ccc2c2cc3c(cc21)c1ccc(-c2ccc(C=C(C#N)C#N)s2)cc1n3CC]

7. The organic photovoltaic cell material according to claim 1, wherein the material is

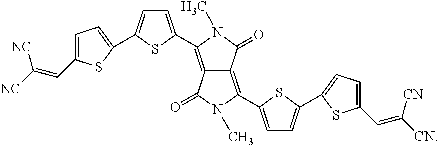 [SMILES: CN1C(=O)C2=C(c3ccc(-c4ccc(C=C(C#N)C#N)s4)s3)N(C)C(=O)C2=C1c1ccc(-c2ccc(C=C(C#N)C#N)s2)s1]

8. An organic photovoltaic cell component, which comprises an electron-donor layer, at least one electron-acceptor layer and an electron transport layer or an hole/exciton barrier layer which are attached to a substrate by the thermal evaporation or the spin coating in turn and which are arranged between an anode and a cathode; wherein the electron-donor layer contains the organic photovoltaic cell material of claim 1.
